(19) United States
(12) Patent Application Publication
(10) Pub. No.: US 2015/0366531 A1
(43) Pub. Date: Dec. 24, 2015

(54) ULTRASONIC DIAGNOSTIC APPARATUS AND METHOD THEREOF

(71) Applicant: Institute for Basic Science, Daejeon (KR)

(72) Inventors: Chi Young Ahn, Daejeon (KR); Ki Wan Jeon, Daejeon (KR); Jung II Choi, Seoul (KR)

(73) Assignee: Institute for Basic Science, Daejeon (KR)

(21) Appl. No.: 14/741,178

(22) Filed: Jun. 16, 2015

(30) Foreign Application Priority Data
Jun. 18, 2014 (KR) 10-2014-0074622

(51) Int. Cl. A61B 8/06; A61B 8/08; A61B 8/14

(52) U.S. Cl. CPC A61B 8/06; A61B 8/14; A61B 8/488; A61B 8/5207; A61B 8/5223; A61B 8/0883

(57) ABSTRACT

Disclosed is an ultrasonic diagnostic apparatus. The ultrasonic diagnostic apparatus includes: an ultrasonic sensor transmitting ultrasonic waves and sensing echo signals thereof; a signal processor processing the signals sensed by the ultrasonic sensor; a B-mode image generation unit generating a brightness mode (B-mode) image in a 2D plane based on the signal processing results by the signal processor and extracting left ventricle boundary data from the generated B-mode image; a C-mode image generation unit generating Doppler data for generation of a C-mode image based on the signal processing results by the signal processor; and a blood flow velocity vector calculation unit calculating a blood flow velocity vector in a 2D image plane based on a simultaneous equation composed of a relation equation between the extracted left ventricle boundary data, the Doppler data and the blood flow velocity vector, and a 2D Navier-Stokes equation obtained from image data generated through signal processing by the signal processor, wherein the Navier-Stokes equation further uses, as a variable, a mass source term representing distribution of source and sink of mass with respect to the 2D image plane.

### BACKGROUND

1. Technical Field

The present invention relates to an ultrasonic diagnostic apparatus and an ultrasonic diagnostic method thereof, and more particularly, to an ultrasonic diagnostic apparatus, which can exactly calculate a velocity vector of a blood flow in the heart or the like using left ventricle boundary data extracted from 2D brightness mode (B-mode) echocardiograms and Doppler data for generation of color Doppler mode (C-mode) images, without injection of contrast agents (or contrast enhancing media) into blood vessels in ultrasonic diagnosis of the heart or the like, and an ultrasonic diagnostic method thereof.

2. Description of the Related Art

Ultrasonic diagnostic apparatuses are widely used in medicine to acquire information on an internal structure of a subject due to their noninvasive and nondestructive characteristics. Further, ultrasonic diagnostic apparatuses are essentially used in medicine by virtue of their capabilities of providing medical doctors with high resolution images of internal organs and tissues of the human body in real time without a need for surgical operations accompanied by direct incision to observe a subject.

In the ultrasonic diagnostic apparatus, a conversion device is electrically stimulated to generate ultrasonic signals, which in turn are transmitted to the human body. The transmitted ultrasonic sonic signals are reflected at boundaries of discontinuous human tissues, and ultrasonic echo signals transmitted from the boundaries to the conversion device are converted into electric signals. The converted electric signals are subjected to amplification and signal processing to generate echocardiogram data.

Quantitative information on the functions of the heart is required for heart disease diagnosis. The quantitative information includes left ventricular hypertrophy, stroke volume, ejection fraction, cardiac output, and the like. In recent years, a lot of attention has been paid to a technique for utilizing vorticity of a blood flow in diagnosis by calculating velocity vectors of the blood flow and quantifying the vorticity of the blood flow based on the calculated velocity vectors. In order to acquire the quantitative information on the functions of the heart, it is necessary to achieve three-dimensional identification of directions and velocities of the blood flow within a left ventricle that shrinks and expands to supply blood to the whole body.

In the related art, directions and velocities of the blood flow within the heart can be mainly identified by a method of calculating blood flow vectors by injecting contrast agents and tracing movement of speckles within the heart.

However, such a technique has disadvantages in that contrast enhancing media are necessarily injected to calculate information on vector components of the blood flow within the heart, and capabilities of tracing speckles of the blood flow seriously depend upon image quality. For this reason, it is difficult to accurately calculate three-dimensional blood flow vector information.

One example of the related art is disclosed in Korean Patent Publication No. 1995-7000029 (published on Jan. 16, 1995).

### BRIEF SUMMARY

Embodiments of the present invention provide an ultrasonic diagnostic apparatus, which can exactly calculate a velocity vector of a blood flow in the heart or the like using left ventricle boundary data extracted from 2D B-mode echocardiograms and Doppler data for generation of C-mode images, without injecting contrast enhancing media into blood vessels in ultrasonic diagnosis of the heart or the like, and an ultrasonic diagnostic method thereof.

In accordance with one aspect of the present invention, an ultrasonic diagnostic apparatus includes: an ultrasonic sensor transmitting ultrasonic waves and sensing echo signals thereof; a signal processor processing the signals sensed by the ultrasonic sensor; a B-mode image generation unit generating a brightness mode (B-mode) image in a 2D plane based on the signal processing results by the signal processor and extracting left ventricle boundary data from the generated B-mode image; a C-mode image generation unit generating Doppler data for generation of a C-mode image based on the signal processing results by the signal processor; and a blood flow velocity vector calculation unit calculating a blood flow velocity vector in a 2D image plane based on a simultaneous equation composed of a relation equation between the extracted left ventricle boundary data, the Doppler data and the blood flow velocity vector, and a 2D Navier-Stokes equation obtained from image data generated through signal processing by the signal processor, wherein the Navier-Stokes equation further uses, as a variable, a mass source term representing distribution of source and sink of mass with respect to the 2D image plane.

The blood flow velocity vector calculation unit calculates the blood flow velocity vector through linearization of the simultaneous equation and discretization of an equation resulting from the linearization.

The discretization is achieved by applying a standard finite difference method to the equation resulting from the linearization.

The Navier-Stokes equation uses a left ventricle boundary extracted from the B-mode image as boundary conditions.

The Doppler data for generation of a C-mode image is obtained by an inner product between a direction vector of an ultrasonic scan line and a velocity vector of an actual blood flow.

In accordance with another aspect of the present invention, an ultrasonic diagnostic method using an ultrasonic diagnostic apparatus includes: transmitting, by an ultrasonic sensor, ultrasonic waves and sensing echo signals thereof; processing the signals sensed by the ultrasonic sensor; generating a B-mode image in a 2D plane based on the signal processing results and extracting left ventricle boundary data from the generated B-mode image; calculating Doppler data for generation of a C-mode image based on the signal processing results; and calculating a blood flow velocity vector in a 2D image plane based on a simultaneous equation composed of a relation equation between the extracted left ventricle boundary data, the Doppler data and the blood flow velocity vector, and a 2D Navier-Stokes equation obtained from image data generated through the signal processing, wherein the Navier-Stokes equation further uses, as a variable, a mass source term representing distribution of source and sink of mass with respect to the 2D image plane.

In calculating a blood flow velocity vector, the blood flow velocity vector may be calculated through linearization of the simultaneous equation and discretization of an equation resulting from the linearization.

The discretization may be achieved by applying a standard finite difference method to the equation resulting from the linearization.

The Navier-Stokes equation may use a left ventricle boundary extracted from the B-mode image as boundary conditions.

The Doppler data for generation of a C-mode image may be obtained by an inner product between a direction vector of an ultrasonic scan line and a velocity vector of an actual blood flow.

The ultrasonic diagnostic apparatus and method according to the present invention can exactly calculate a velocity vector of a blood flow in the heart or the like using a 2D Navier-Stokes equation employing a mass source term representing distribution of source and sink of mass with respect to a 2D image plane as an additional variable, without a need for injecting contrast enhancing media into blood vessels in diagnosis of the heart or the like.

In addition, the ultrasonic diagnostic apparatus and method according to the present invention uses existing Doppler data for C-mode images and can thus be implemented only by software processing through a simple arithmetic operation without changing hardware design.

### BRIEF DESCRIPTION OF THE DRAWINGS

The above and other aspects, features, and advantages of the present invention will become apparent from the detailed description of the following embodiments in conjunction with the accompanying drawings, in which:

FIG. 1 is a view showing features of an ultrasonic diagnostic apparatus according to one embodiment of the present invention;

FIG. 2 is a view showing a 2-chamber view, a 3-chamber view, and a 4-chamber view according to scan angle in cardiac scanning using the ultrasonic diagnostic apparatus;

FIG. 3 is a conceptual view of a blood flow velocity vector according to blood flow stream on the 3-chamber view of the images in FIG. 2;

FIG. 4 is a view showing a vector of an ultrasonic scan line in the ultrasonic diagnostic apparatus; and

FIG. 5 is a flowchart illustrating an ultrasonic diagnostic method according to one embodiment of the present invention.

### DETAILED DESCRIPTION

Hereinafter, exemplary embodiments of the present invention will be described in detail with reference to the accompanying drawings. The following embodiments are given by way of illustration to provide a thorough understanding of the disclosure to those skilled in the art. It should be understood that the present invention is not limited to the following embodiments and may be embodied in different ways. In the drawings, portions irrelevant to the description will be omitted for clarity. Like components will be denoted by like reference numerals throughout the specification.

It will be understood that the terms “comprise,” “include,” and/or “have(has)” as used in this specification, specify the presence of stated features, steps, operations, elements, and/or components, but do not preclude the presence or addition of one or more other features, steps, operations, elements, components, and/or groups.

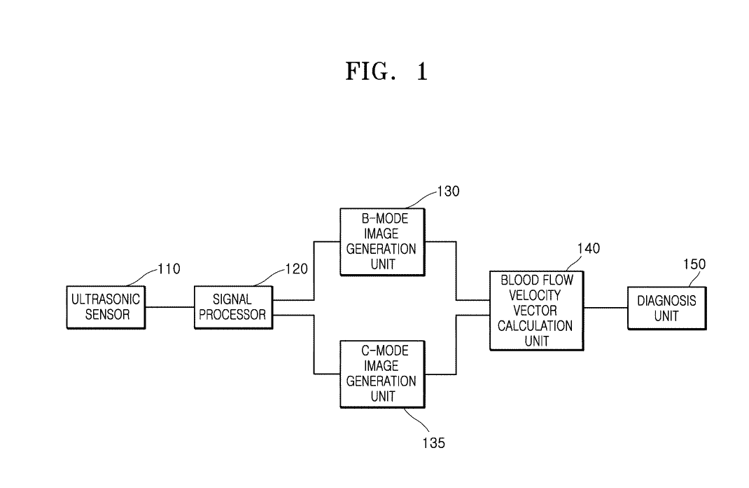

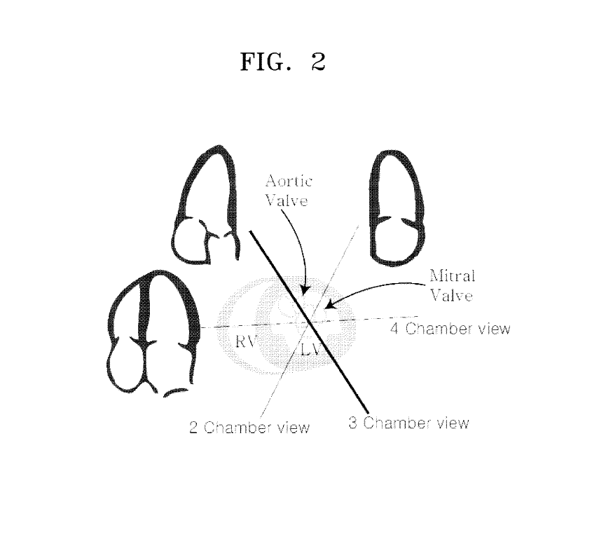

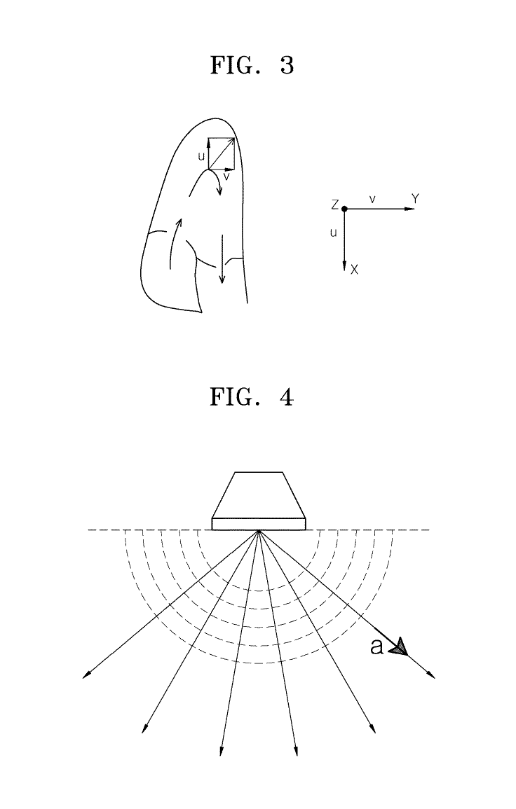

FIG. 1 is a view showing features of an ultrasonic diagnostic apparatus according to one embodiment of the present invention; FIG. 2 is a view showing a 2-chamber view, a 3-chamber view, and a 4-chamber view according to scan angle in cardiac scanning using the ultrasonic diagnostic apparatus; FIG. 3 is a conceptual view of a blood flow velocity vector according to blood flow stream on the 3-chamber view of the images in FIG. 2; and FIG. 4 is a view showing a vector of an ultrasonic scan line in the ultrasonic diagnostic apparatus.

Referring to FIG. 1, an ultrasonic diagnostic apparatus according to the embodiment includes an ultrasonic sensor 110, a signal processor 120, a B-mode image generation unit 130, a C-mode image generation unit 135, a blood flow velocity vector calculation unit 140, and a diagnosis unit 150.

The ultrasonic sensor 110 transmits ultrasonic waves to a subject, particularly, a left ventricle of the heart, and senses echo signals thereof.

The signal processor 120 processes the signals sensed by the ultrasonic sensor 110. Specifically, the signal processor conducts signal processing, based on which the ultrasonic diagnostic apparatus performs various operations such as realization and diagnosis of echocardiograms using the sensed ultrasonic signals.

The B-mode image generation unit 130 generates a brightness mode (B-mode) image in a 2D plane based on signal processing results by the signal processor 120 and extracts left ventricle boundary data from the B-mode image for an entire cardiac cycle.

The C-mode image generation unit 135 calculates Doppler data (color Doppler data) for generation of a C-mode image in a 2D image plane based on the signal processing results by the signal processor 120. Although the present invention is described by way of example wherein the C-mode image generation unit 135 serves to both calculate Doppler data for generation of a C-mode image and to generate the C-mode image based on the Doppler data, the present invention is not limited thereto. Thus, a separate component for calculating the Doppler data and a component for generating the C-mode image may also be provided.

The blood flow velocity vector calculation unit 140 calculates a blood flow velocity vector in a 2D image plane based on a simultaneous equation composed of a relation equation between the left ventricle boundary data extracted from the B-mode image, the Doppler data and the blood flow velocity vector, and a 2D Navier-Stokes equation obtained from image data generated through signal processing of the signal processor 120. Here, the Navier-Stokes equation further uses, as a variable, a mass source term which represents distribution of source and sink of mass with respect to the 2D image plane.

Particularly, the blood flow velocity vector calculation unit 140 calculates the blood flow velocity vector based on linearization of the simultaneous equation and discretization of an equation resulting from the linearization, wherein the discretization is achieved by applying a standard finite difference method to the equation resulting from the linearization.

In this embodiment, the simultaneous equation including the Navier-Stokes equation uses the left ventricle boundary as boundary conditions.

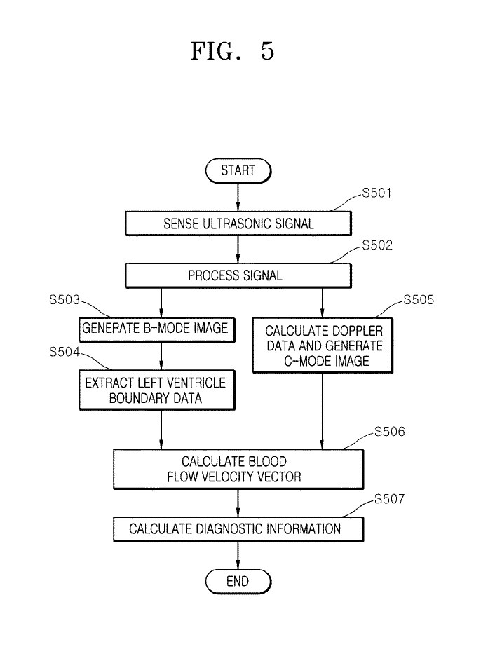

Operations and functions of this embodiment will be described in detail with reference to FIGS. 1 to 5. FIG. 5 is a flowchart illustrating an ultrasonic diagnostic method according to one embodiment of the present invention.

First, as shown in FIG. 5, the ultrasonic sensor 110 transmits ultrasonic waves and senses echo signals thereof, and outputs the sensed signals (S501). Although the ultrasonic sensor 110 may perform an ultrasonic wave sensing operation on a subject, particularly, the left ventricle of the heart, in this embodiment, the present invention is not limited thereto.

Next, the signal processor 120 processes the signals sensed by the ultrasonic sensor 110 (S502). The signal processor 120 conducts signal processing, based on which the ultrasonic diagnostic apparatus performs various operations such as realization and diagnosis of echocardiograms using the sensed ultrasonic signals. Since specific operations of the signal processor are the same as in a typical ultrasonic diagnostic apparatus, detailed descriptions thereof will be omitted.

FIG. 2 is a view showing a 2-chamber view, a 3-chamber view, and a 4-chamber view according to scan angle in cardiac scanning using the ultrasonic diagnostic apparatus. The echocardiograms according to scan direction as shown in FIG. 2 may be generated using signal processing results by the signal processor 120. In particular, FIG. 3 is a conceptual view of a blood flow velocity vector according to blood flow stream in the 3-chamber view of the images in FIG. 2. The blood flow velocity vector (u, v) in a 2D image plane may be conceptually expressed as in FIG. 3 according to time and position.

Next, the B-mode image generation unit 130 generates a B-mode image in a 2D plane based on the signal processing results by the signal processor 120 (S503) and extracts left ventricle boundary data from the generated B-mode image (S504). The brightness mode (B-mode) image is obtained by a process in which reflection coefficients of ultrasonic signals (i.e. ultrasonic echo signals) reflected from a subject are transformed into a 2D image. Since a technique of generating the B-mode image is well known in the art, a detailed description thereof will be omitted. The B-mode image generation unit 130 may extract boundary data for the left ventricle from the generated B-mode image. Here, the 2D image plane refers to a plane corresponding to a 2D echocardiogram obtained by the ultrasonic diagnostic apparatus, and extraction of the left ventricle boundary data is achieved using an image processing algorithm for segmentation and tracking of echocardiograms.

The C-mode image generation unit 135 calculates Doppler data (color Doppler data) in the 2D image plane for generation of a C-mode image based on the signal processing results by the signal processor 120, thereby generating the C-mode image (S505). The color Doppler mode (C-mode) image is an image obtained by a process in which speed of a moving subject is expressed by color using the Doppler effect. The C-mode image generation unit 135 generates Doppler data (Doppler signals) using data resulting from signal processing by the signal processor 120 and generates the C-mode image based on the generated Doppler data. Since a technique of generating the C-mode image is well known in the art, a detailed description thereof will be omitted.

Referring to FIG. 4, when an ultrasonic scan line vector according to scan direction in the ultrasonic diagnostic apparatus is a, the Doppler data can be obtained by Equation 1:

c(x,t)(a₁(x),a₂(x))·(u(x,t),v(x,t)).

Here, c(x, t) is Doppler data at a specific point x in the 2D image plane D and time t; (a₁(x), a₂(x)) is an ultrasonic scan line vector a; and (u(x, t), v(x, t)) (hereinafter, referred to as “(u, v)”) is a blood flow velocity vector in the 2D image plane. Here, u and v are x and y components of the blood flow velocity vector, respectively. Namely, as can be seen in Equation 1, c(x, t) is obtained by an inner product between the ultrasonic scan line vector a and the blood flow velocity vector, and is thus a velocity component in a scan line direction.

Next, the blood flow vector calculation unit 140 calculates the blood flow velocity vector in the 2D image plane based on a simultaneous equation composed of a relation equation between the extracted left ventricle boundary data, the Doppler data and the blood flow velocity vector, and a 2D Navier-Stokes equation obtained from image data generated through signal processing of the signal processor 120 (S506).

The Navier-Stokes equation is a non-linear partial differential equation describing fluid dynamics, and it has been known that movement of fluid, including vortices and eddies, can be modeled by applying boundary conditions to the Navier-Stokes equation. In this embodiment, the Navier-Stokes equation uses the left ventricle boundary as boundary conditions. The Navier-Stokes equation for calculation of the blood flow vectors in the left ventricle may be expressed by Equation 2:

$$\{ \begin{matrix}ρ ( \frac{\partial v}{\partial t} + v \cdot ∇ v ) = - ∇ p + μ {∇}^{2} v in {Ω}_{T} , \\ ∇ \cdot v = 0 in {Ω}_{T} , \end{matrix}$$

where the vector v=(u, v, w) is a 3D blood flow velocity vector; p is the density of the blood flow; μ is the coefficient of viscosity of the blood flow, and p is the pressure. Here, u, v, and w are x, y, and z components of the blood flow velocity vector, respectively, Ω_{T} denotes a time-varying left ventricular region and is defined as

$${Ω}_{T} \text{:=} {⋃}_{0 < t < T} Ω ( t ) \times \{ t \} .$$

Here, Ω(t) is a left ventricular region changeable with time, and T is cardiac cycle. Equation 2 is a 3D Navier-Stokes equation using the 3D blood flow velocity vector v.

In order to obtain the blood flow velocity vector (u, v) in the 2D image plane, rewriting in terms of the velocity vector term relating to the 2D plane, Equation 2 may be expressed by Equation 3:

$$\{ \begin{matrix}\frac{\partial u}{\partial t} + u \frac{\partial u}{\partial x} + v \frac{\partial u}{\partial y} = - \frac{1}{ρ} \frac{\partial p}{\partial x} + \frac{μ}{ρ} ( \frac{{\partial }^{2} u}{\partial {x}^{2}} + \frac{{\partial }^{2} u}{\partial {y}^{2}} ) + {\frac{μ}{ρ} \frac{{\partial }^{2} u}{\partial {z}^{2}} - w \frac{\partial u}{\partial z}}_{\begin{matrix}⏟ \\ {f}_{1}\end{matrix}} , \\ \frac{\partial v}{\partial t} + u \frac{\partial v}{\partial x} + v \frac{\partial v}{\partial y} = - \frac{1}{ρ} \frac{\partial p}{\partial y} + \frac{μ}{ρ} ( \frac{{\partial }^{2} v}{\partial {x}^{2}} + \frac{{\partial }^{2} v}{\partial {y}^{2}} ) + {\frac{μ}{ρ} \frac{{\partial }^{2} v}{\partial {z}^{2}} - w \frac{\partial v}{\partial z} ,}_{\begin{matrix}⏟ \\ {f}_{2}\end{matrix}} \\ \frac{\partial u}{\partial x} + \frac{\partial v}{\partial y} = - \frac{\partial w}{\partial z} . \end{matrix}$$

However, the Doppler data c(x, t) obtained by Equation 1 are acquired with respect to the 2D image plane D and do not include information on f₁, f₂, and

$$- \frac{\partial w}{\partial z}$$

of Equation 2 in the 2D image plane D. Thus, in this embodiment, a new variable s, i.e. a mass source term, is employed, whereby Equation 4 can be drawn from Equation 3.

$$\{ \begin{matrix}\frac{\partial u}{\partial t} + u \frac{\partial u}{\partial x} + v \frac{\partial u}{\partial y} = - \frac{1}{ρ} \frac{\partial p}{\partial x} + \frac{μ}{ρ} {∇}^{2} u + \frac{μ}{3 {ρ}^{2}} \frac{\partial s}{\partial x} , \\ \frac{\partial v}{\partial t} + u \frac{\partial v}{\partial x} + v \frac{\partial v}{\partial y} = - \frac{1}{ρ} \frac{\partial p}{\partial y} + \frac{μ}{ρ} {∇}^{2} v + \frac{μ}{3 {ρ}^{2}} \frac{\partial s}{\partial y} , \\ \frac{\partial u}{\partial x} + \frac{\partial v}{\partial y} = \frac{s}{ρ} . \end{matrix} \qquad [ Equation 4 ]$$

The mass source term s is a term representing distribution of source and sink of mass with respect to the 2D image plane, wherein inflow of a fluid to the 2D image plane is defined as source, and outflow of a fluid from the 2D image plane is defined as sink.

Simultaneous equations of Equation 2 and Equation 4 obtained as above are given by Equation 5 through linearization thereof.

$$[ \begin{matrix}{a}_{1} & {a}_{2} & 0 & 0 \\ \frac{\partial }{\partial x} & \frac{\partial }{\partial y} & 0 & - \frac{1}{ρ} \\ \frac{\partial }{\partial t} - \frac{μ}{ρ} {∇}^{2} & 0 & \frac{1}{ρ} \frac{\partial }{\partial x} & - \frac{μ}{3 {ρ}^{2}} \frac{\partial }{\partial x} \\ 0 & \frac{\partial }{\partial t} - \frac{μ}{ρ} {∇}^{2} & \frac{1}{ρ} \frac{\partial }{\partial y} & - \frac{μ}{3 {ρ}^{2}} \frac{\partial }{\partial y}\end{matrix} ] [ \begin{matrix}u \\ v \\ p \\ s\end{matrix} ] = [ \begin{matrix}c \\ 0 \\ - u \frac{\partial u}{\partial x} - v \frac{\partial u}{\partial y} \\ - u \frac{\partial v}{\partial x} - v \frac{\partial v}{\partial y}\end{matrix} ]$$

Further, this equation resulting from the linearization is subjected to discretization, thereby obtaining Equation 6. Here, the discretization is achieved by applying a standard finite difference method to the equation resulting from the linearization.

$$[ \begin{matrix}{a}_{1} & {a}_{2} & 0 & 0 \\ {D}_{x} & {D}_{y} & 0 & - \frac{1}{ρ} \\ 1 - \frac{μΔ t}{ρ} D & 0 & \frac{Δ t}{ρ} {D}_{x} & - \frac{μΔ t}{3 {ρ}^{2}} {D}_{x} \\ 0 & 1 - \frac{μΔ t}{ρ} L & \frac{Δ L}{ρ} {D}_{y} & - \frac{μΔ t}{3 {ρ}^{2}} {D}_{y}\end{matrix} ] [ \begin{matrix}{u}^{( n + 1 )} \\ {v}^{( n + 1 )} \\ {p}^{( n + 1 )} \\ {s}^{( n + 1 )}\end{matrix} ] = [ \begin{matrix}{c}^{( n + 1 )} \\ 0 \\ {u}^{( n )} - Δ t ( {u}^{( n )} {D}_{x} {u}^{( n )} + {v}^{( n )} {D}_{y} {u}^{( n )} ) \\ {v}^{( n )} - Δ t ( {u}^{( n )} {D}_{x} {v}^{( n )} + {v}^{( n )} {D}_{y} {v}^{( n )} )\end{matrix} ] \qquad [ Equation 6 ]$$

where Dx denotes x-derivative, Dy denotes y-derivative, L denotes Laplace operator, and (n) denotes the n^{th} time step.

The blood flow velocity vector calculation unit 140 may compute a recurrence equation such as Equation 6 within the left ventricle boundary extracted from each of B-mode images for an entire cardiac cycle to obtain u and v falling within a reasonable margin of error, thereby obtaining the blood flow velocity vector (u, v) in the 2D image plane. In Equation 6, an initial value of the blood flow velocity vector (u, v) in the 2D image plane may be set to a value of u=0 and v=0 or u=c and v=0 (c is the Doppler data in Equation 1), without being limited thereto.

Next, the diagnosis unit 150 calculates quantitative diagnostic information on heart function, such as vorticity of blood flow, based on the blood flow velocity vector in the 2D image plane calculated by the blood flow velocity vector calculation unit 140.

As described above, the ultrasonic diagnostic apparatus and method according to the present invention can exactly calculate a velocity vector of a blood flow in the heart or the like using the 2D Navier-Stokes equation employing a mass source term representing distribution of source and sink of mass with respect to a 2D image plane as an additional variable, without a need for injecting contrast enhancing media into blood vessels in diagnosis of the heart or the like. In addition, the ultrasonic diagnostic apparatus and method according to the present invention use existing Doppler data for C-mode images and can thus be implemented only by software processing through a simple arithmetic operation without changing hardware design. Although some embodiments have been described it should be understood that these embodiments are provided for illustration only and that various modifications and other equivalent embodiments can be made without departing from the spirit and the scope of the present invention. Thus, the scope of the present invention should be determined by the attached claims.

### LEGEND OF REFERENCE NUMERALS

110: Ultrasonic sensor
120: Signal processor
130: B-mode image generation unit
135: C-mode image generation unit
140: Blood flow velocity vector calculation unit
150: Diagnosis unit

What is claimed is:

1. An ultrasonic diagnostic apparatus comprising:
an ultrasonic sensor transmitting ultrasonic waves and sensing echo signals thereof;
a signal processor processing the signals sensed by the ultrasonic sensor;
a B-mode image generation unit generating a brightness mode (B-mode) image in a 2D plane based on the signal processing results by the signal processor and extracting left ventricle boundary data from the generated B-mode image;
a C-mode image generation unit generating Doppler data for generation of a C-mode image based on the signal processing results by the signal processor; and
a blood flow velocity vector calculation unit calculating a blood flow velocity vector in a 2D image plane based on a simultaneous equation composed of a relation equation between the extracted left ventricle boundary data, the Doppler data and the blood flow velocity vector, and a 2D Navier-Stokes equation obtained from image data generated through signal processing by the signal processor,
wherein the Navier-Stokes equation further uses, as a variable, a mass source term representing distribution of source and sink of mass with respect to the 2D image plane.

2. The ultrasonic diagnostic apparatus according to claim 1, wherein the blood flow velocity vector calculation unit calculates the blood flow velocity vector through linearization of the simultaneous equation and discretization of an equation resulting from the linearization.

3. The ultrasonic diagnostic apparatus according to claim 2, wherein the discretization is achieved by applying a standard finite difference method to the equation resulting from the linearization.

4. The ultrasonic diagnostic apparatus according to claim 1, wherein the simultaneous equation composed of the relation equation between the left ventricle boundary data, the Doppler data and the blood flow velocity vector, and the 2D Navier-Stokes equation uses a left ventricle boundary extracted from the B-mode image as boundary conditions.

5. The ultrasonic diagnostic apparatus according to claim 1, wherein the Doppler data for generation of a C-mode image is obtained by an inner product between a direction vector of ultrasonic scan line and a velocity vector of an actual blood flow.

6. An ultrasonic diagnostic method using an ultrasonic diagnostic apparatus, comprising:
transmitting, by an ultrasonic sensor, ultrasonic waves and sensing echo signals thereof;
processing the signals sensed by the ultrasonic sensor;
generating a B-mode image in a 2D plane based on the signal processing results and extracting left ventricle boundary data from the generated B-mode image;
calculating Doppler data for generation of a C-mode image based on the signal processing results; and
calculating a blood flow velocity vector in a 2D image plane based on a simultaneous equation composed of a relation equation between the extracted left ventricle boundary data, the Doppler data and the blood flow velocity vector, and a 2D Navier-Stokes equation obtained from image data generated through the signal processing,
wherein the Navier-Stokes equation further uses, as a variable, a mass source term representing distribution of source and sink of mass with respect to the 2D image plane.

7. The ultrasonic diagnostic method according to claim 6, wherein, in calculating a blood flow velocity vector, the blood flow velocity vector is calculated through linearization of the simultaneous equation and discretization of an equation resulting from the linearization.

8. The ultrasonic diagnostic method according to claim 7, wherein the discretization is achieved by applying a standard finite difference method to the equation resulting from the linearization.

9. The ultrasonic diagnostic method according to claim 6, wherein the simultaneous equation composed of the relation equation between the left ventricle boundary data, the Doppler data and the blood flow velocity vector, and the 2D Navier-Stokes equation uses a left ventricle boundary extracted from the B-mode image as boundary conditions.

10. The ultrasonic diagnostic method according to claim 6, wherein the Doppler data for generation of a C-mode image is obtained by an inner product between a direction vector of an ultrasonic scan line and a velocity vector of an actual blood flow.
